(19) United States
(12) Patent Application Publication
(10) Pub. No.: US 2025/0054072 A1
(43) Pub. Date: Feb. 13, 2025

(54) METHOD AND SYSTEM FOR PARTNER COMPATIBILITY MATCHING

(71) Applicant: Maier J. Tarlow, Brooklyn, NY (US)

(72) Inventor: Maier J. Tarlow, Brooklyn, NY (US)

(21) Appl. No.: 18/797,438

(22) Filed: Aug. 7, 2024

Related U.S. Application Data
(60) Provisional application No. 63/532,420, filed on Aug. 13, 2023.

(51) Int. Cl. G06Q 50/00; G16H 10/65; H04L 67/306

(52) U.S. Cl. CPC G06Q 50/01; G16H 10/65; H04L 67/306

(57) ABSTRACT

A method includes receiving, in association with a profile, an electronic transfer having a data structure and including an identification of the transmitter; determining, by a processor, that the transmitter is included in a predefined set of transmitters; performing character processing of the data structure, thereby identifying one or more of predefined diseases and respective positivity statuses; conditional upon the inclusion in the set of transmitters, associating the respective identified positivity statuses for the respective diseases with the profile on a central server; and subsequently, responsive to a trigger on a device on which the profile is active: activating an image sensor of the device to scan a compatibility tag; processing the scanned compatibility tag, thereby obtaining a respective positivity status for each of the diseases; and based on the positivity statuses of the compatibility tag and of the profile, rendering a compatibility indicator on a display of the device.

### CROSS-REFERENCE TO RELATED APPLICATIONS

The present invention claims the benefit of U.S. Provisional Patent Application Ser. No. 63/532,420 filed Aug. 13, 2023, the entire content of which is incorporated herein by reference.

### FIELD OF THE INVENTION

The present invention relates to a system and methods for using devices to match user profiles based on positivity statuses with respect to diseases.

### BACKGROUND

Systems are used for matching system users to each other based on profiles. However, ultimately respective medical histories can impact relationships between the users. It therefore can be important for prospective matched users to know specifics of the other matched user before beginning a relationship. However, such medical information is often considered private and divulging such information can be considered embarrassing.

### SUMMARY

Example embodiments of the present invention are directed to user profile matching systems that specify matches in a manner that is safe and maintains medical conditions private.

According to an example embodiment of the present invention, a method includes receiving, by a processor and from a transmitter via a network, at least one electronic transfer having attached thereto a data structure. The electronic transfer includes an identification of the transmitter. The receipt of the electronic transfer is made in association with a first user profile. The processor compares the identified transmitter to a predetermined set of transmitters, thereby determining that the transmitter is included in the set of transmitters.

The processor performs character processing on the attached data structure, thereby identifying in the data structure one or more of a plurality of predefined diseases and, for each of the one or more of the plurality of predefined diseases, a respective positivity status. A positivity status indicates whether one is found to be positive or negative for the respective disease with which the positivity status is associated. For each of the one or more of the plurality of diseases, the processor associates the respective identified positivity status for the respective one of the diseases with the first user profile, in association with which the electronic transfer was made, on a central server.

In an example embodiment, the association of the positivity statuses with the first user profile is made conditional upon the determination of the inclusion of the identified transmitter in the set of transmitters, since this determination verifies the veracity of the positivity statuses. In an alternative embodiment, the association can be made even without the determination regarding the transmitter, but the system flags such an association as unverified.

Subsequent to the association and responsive to a scan trigger on a device on which the first user profile is active, the device activates an image sensor of the device to scan a first compatibility tag, e.g., upon manipulation of a user input component of the device. The processor, e.g., of the device (or of the server after a transmission from the device to the server) processes the scanned first compatibility tag, thereby obtaining a respective positivity status of a second user profile for each of the plurality of diseases. Such statuses can include negative statuses, positive statuses, or NULL statuses or the like in case the second user profile does not have a populated positivity status for any of the diseases. Additionally, in an example embodiment, further details can be obtained for the respective statuses, such as whether the respective statuses are verified.

Based on a combination of the positivity statuses (of the second user profile) obtained from the processing of the scanned first compatibility tag and the respective identified positivity status for the one or more of the plurality of diseases associated with the first user profile, the device or the server generates a compatibility indicator, and the processor of the device renders the compatibility indicator on a display of the device. The compatibility indicator identifies whether the first and second user profiles are compatible with respect to safety.

In an example, the method further includes the server associating the receipt of the electronic transfer with the first user profile based on the transmission being addressed with an electronic recipient address that is associated with the first user profile.

In an example, the first compatibility tag is a Quick Response (QR) code.

In an example, the method further includes, subsequent to the association, generating, by the device on which the user profile is active or by the server, a second compatibility tag, where the second compatibility tag is processable by a second device to obtain, by the second device, the respective positivity status for the one or more diseases associated with the first user profile. The second compatibility tag is then rendered the display of the device, so that the second device can scan the rendered compatibility tag. In this way the first and second devices can each be used to scan and process compatibility tags of the other device, so that users of each of the two devices can obtain respective compatibility indicators pertaining to the respective user profiles active on the other of the devices.

In an example, the generating and rendering of the second compatibility tag occurs responsive to the scan trigger. For example, in response to the scan trigger on the first device, the first device scans the first compatibility tag on the display of the second device and also displays the second compatibility tag on the first device for scanning by the second device.

In an alternative example, the generating and rendering of the second compatibility tag occurs responsive to a tag generating trigger separate from the scan trigger.

In an example, where the compatibility indicator is set to positive, which indicates that there is compatibility, the setting is made conditional upon that the positivity statuses obtained by the processing of the scanned first compatibility tag do not include a positive status (with respect to the second user profile) for any disease for which a negative status is associated with the first user profile.

In an example, where the compatibility indicator is set to positive, the setting is made conditional upon that the positivity statuses obtained by the processing of the scanned first compatibility tag do not include a positive status (with respect to the second user profile) for any disease for which a positive status is not associated with the first user profile (even if there is also not a negative status associated with the first user profile).

In an example, where the compatibility indicator is set to negative, the setting is made conditional upon that the positivity statuses obtained by the processing of the scanned first compatibility tag include a positive status for at least one disease for which a negative status (or alternatively not a positive status) is associated with the first user profile, since a joining of the users associated with these user profiles has the potential of harming the user associated with the first user profile.

In an alternative example, where the compatibility indicator is set to negative, the setting is made conditional upon that, for each of at least one disease for which a negative status (or alternatively not a positive status) is associated with the first user profile, the positivity statuses obtained by the processing of the scanned first compatibility tag either include a positive status or do not include a negative status.

In an example, where the compatibility indicator is set to neutral, the setting is made in response to satisfaction of a condition that, for each of at least one disease for which a negative status is associated with the first user profile, the positivity statuses obtained by the processing of the scanned first compatibility tag is that a status has not been provided, e.g., is a NULL field.

In an example, where the compatibility indicator is set to neutral or caution or the like, the setting is made in response to satisfaction of a condition that, for each of at least one disease for which a negative status is associated with the first user profile, the positivity statuses obtained by the processing of the scanned first compatibility tag include a negative status associated with a date that is prior to a current date by a time period that is greater than a predefined time period to which the device compares the time period of the associated date.

In an example, based on the processing of the scanned first compatibility tag, a respective date is obtained by the device of the central server in association with each of the respective positivity statuses obtained from the processing of the scanned first compatibility tag, and the generation of the compatibility indicator is based on, for each of at least one of the respective dates, a comparison of a time period from the respective date until a current date to a predefined time period.

According to an example embodiment of the present invention, a method includes, in response to a first scan trigger on a first device on which a first user profile is active, the device activates an image sensor to record an image. The device or a central server to which the device is communicatively coupled processes the image, thereby extracting one or more of a plurality of predefined diseases and, for each of the one or more of the plurality of predefined diseases, a respective positivity status. In response to the extraction, for each of the one or more of the plurality of diseases, the respective identified positivity status for the respective one of the diseases is associated with the first user profile on the central server. For example, a user may present a document in a visual range of the image sensor for the device to be able to obtain the image, which is then processed to extract the information. For example the document can include a listing of the diseases and test results in respective rows, or may include a QR code or the like from which the data can be extracted.

Subsequent to the association and responsive to a second scan trigger on the first device on which the first user profile is active or on a second device on which the first user profile is active, the first or second device activates an image sensor of the respective first or second device to scan a first compatibility tag.

The respective first or second device or the central server processes the scanned first compatibility tag, thereby obtaining a respective positivity status for each of the plurality of diseases.

Based on a combination of the positivity statuses obtained from the processing of the scanned first compatibility tag and the respective identified positivity status for the one or more of the plurality of diseases associated with the first user profile, the respective first or second device or the server generates a compatibility indicator and the compatibility indicator is rendered on a display of the respective first or second device.

In an example, the processing of the image includes extracting an identification of an entity. The method further includes comparing, by the device or the server, the identified entity to a predetermined set of entities, thereby determining that the identified entity is included in the set of entities, and the associating of the respective identified positivity status for the each respective one of the diseases with the first user profile on the server is performed conditional upon the determination of the inclusion of the identified entity in the set of entities, according to an example in which verification is required.

According to an example embodiment of the present invention, a system includes a central server and a first device that is communicatively coupled to the central server via a network. The first device includes an image sensor, a display, and a processor.

The central server is configured to receive, from a transmitter and via the network, at least one electronic transfer having attached thereto a data structure, the electronic transfer including an identification of the transmitter, the receipt being made in association with a first user profile. The central server is configured to compare the identified transmitter to a predetermined set of transmitters, thereby determining that the transmitter is included in the set of transmitters. The central server is configured to perform a character processing of the attached data structure, thereby identifying in the data structure one or more of a plurality of predefined diseases and, for each of the one or more of the plurality of predefined diseases, a respective positivity status. Conditional upon the determination of the inclusion of the identified transmitter in the set of transmitters, for each of the one or more of the plurality of diseases, the central server is configured to associate the respective identified positivity status for the respective one of the diseases with the first user profile.

Subsequent to the association and responsive to a scan trigger on the first device on which the first user profile is active by a communication of the device with the server, the processor of the first device is configured to activate the image sensor of the first device to scan a first compatibility tag. The central server or the processor of the first device is configured to process the scanned first compatibility tag, thereby obtaining a respective positivity status for each of the plurality of diseases. Based on a combination of the positivity statuses obtained from the processing of the scanned first compatibility tag and the respective identified positivity status for the one or more of the plurality of diseases associated with the first user profile, the processor of the first device or the server is configured to generate a compatibility indicator. While the first user profile is active on the first device, the processor is configured to render the compatibility indicator on the display of the first device.

According to an example embodiment of the present invention, a method includes, in response to an obtainment trigger on a first device on which a first user profile is active, the device of a central server to which the device is communicatively coupled, processes a data structure, thereby extracting one or more of a plurality of predefined diseases and, for each of the one or more of the plurality of predefined diseases, a respective positivity status. For example, the data structure may be obtained from a local or external storage to which it had previously been uploaded or may be obtained by performing scanning.

In response to the extraction, for each of the one or more of the plurality of diseases, the respective identified positivity status for the respective one of the diseases is associated with the first user profile on the central server.

Subsequent to the association and responsive to a scan trigger on the first device on which the first user profile is active or on a second device on which the first user profile is active, the first or second device activates an image sensor of the respective first or second device to scan a first compatibility tag. The respective first or second device or the central server processes the scanned first compatibility tag, thereby obtaining a respective positivity status for each of the plurality of diseases. Based on a combination of the positivity statuses obtained from the processing of the scanned first compatibility tag and the respective identified positivity status for the one or more of the plurality of diseases associated with the first user profile, the respective first or second device or the server generates a compatibility indicator, which is rendered on a display of the respective first or second device.

According to an example embodiment of the present invention, a method includes, for each of one or more of a plurality of diseases, associating, by the central server, a respective positivity status for the respective disease with a first user profile. Subsequent to the association and responsive to a scan trigger on a device on which the first user profile is active and that is communicatively coupled to the central server, the device activates an image sensor of the device to scan a first compatibility tag, for example that is displayed on another device. The first device of the central server processes the scanned first compatibility tag, thereby obtaining a respective scanned positivity status for each of the plurality of diseases. Based on a combination of the positivity statuses obtained from the processing of the scanned first compatibility tag and the respective positivity status for the one or more of the plurality of diseases associated with the first user profile, the device or the central server generates a compatibility indicator. The generated compatibility indicator is then rendered on a display of the device.

According to an example embodiment of the present invention, a method includes, for each of a plurality of user profiles, associating, by a central server and with the respective user profile, a positivity status for each disease of a respective subset of a plurality of diseases. Subsequent to the associations, the central processor performs pairwise matching of respective pairs of the plurality of user profiles at least partially based on the positivity statuses associated with the plurality of user profiles. For each of the pairs that are matched, the pairing is made conditional upon that the positivity statuses associated with either one of the user profiles of the pair do not include a positive status for any disease for which a negative status is associated with the other user profile of the pair. For each of the respective pairs, each one of the user profiles of the pair is associated with a respective electronic flag linking the other user profile of the pair as a match. Additionally, for each of the respective pairs, responsive to the associations with the respective electronic flags, on an output component of a respective device on which a respective one of the user profiles is active, an indicator of the other user profile or the pair as being a match is generated. Example embodiments of the present invention are explained below in more detail on the basis of the accompanying drawings.

### BRIEF DESCRIPTION OF THE DRAWINGS

FIG. 1 shows system according to an example embodiment of the present invention.

FIG. 2 illustrates devices of the system interacting with each other.

FIGS. 3 and 4 are flowcharts illustrating methods for devices to be used for determining compatibility between user profiles.

FIG. 5 is a flowchart illustrating a method for proactive compatibility matching.

### DETAILED DESCRIPTION

In the following description of preferred example embodiments of the present invention, identical or similar reference characters are used for elements shown in the various figures that have similar function, and repeated description of these elements is not given.

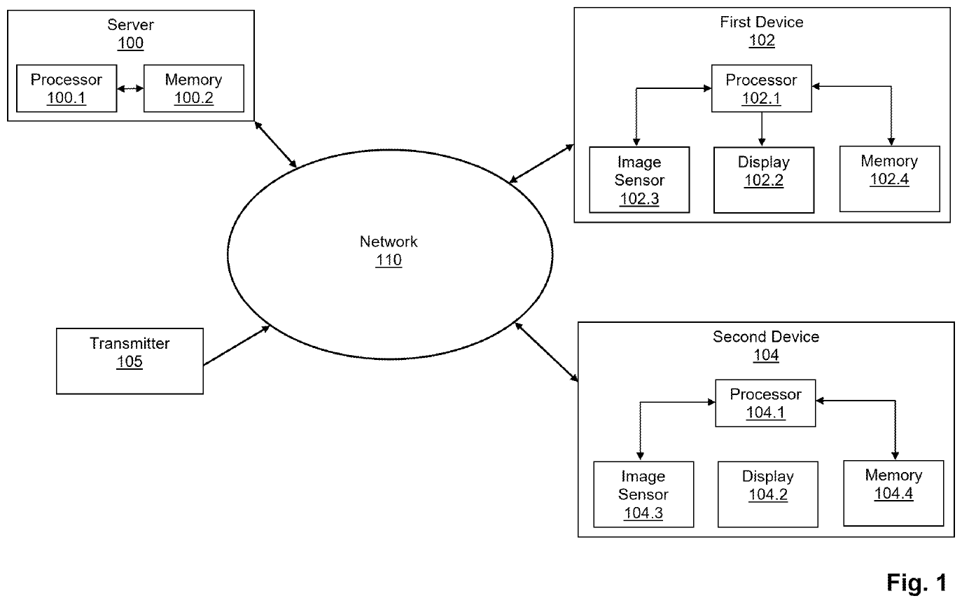

FIG. 1 is a schematic diagram of a system according to an example embodiment of the present invention. The system includes server 100 communicatively coupled to a plurality of devices over a network 110, such as the Internet or other wide area network using wired and/or wireless device interconnections. FIG. 1 shows, for illustrative purposes, two of the plurality of devices, the two of the plurality of devices being a first device 102 and a second device 104. First and second devices 102 and 104 can be in the form of, for example, a desktop computer, a laptop computer, a hand-held device such as a personal digital assistant (PDA), mobile phone, smart phone, or other computing device that is user operable.

The server 100 can include a processor 100.1 and a memory device 100.2. The processor can be implemented using any conventional processing circuit and device or combination thereof, e.g., a Central Processing Unit (CPU) of a Personal Computer (PC) or other workstation or other computer, to execute code provided, e.g., on a hardware computer-readable medium including any conventional memory device, to perform any of the methods described herein with respect to the server, alone or in combination. The memory device 100.2 can include any conventional permanent and/or temporary memory circuits or combination thereof, a non-exhaustive list of which includes Random Access Memory (RAM), Read Only Memory (ROM), Compact Disks (CD), Digital Versatile Disk (DVD), and magnetic tape.

The first device 102 can include a processor 102.1, a display 102.2, an image sensor 102.3, and a memory 102.4. Similarly, the second device 104 can include a processor 104.1, a display 104.2, an image sensor 104.3, and a memory 104.4. The processors 102.1 and 104.1 can be implemented using any conventional processing circuit and device or combination thereof, e.g., a Central Processing Unit (CPU) of a Personal Computer (PC) or other workstation or other computer, to execute code provided, e.g., on a hardware computer-readable medium including any conventional memory device, to perform any of the methods described herein with respect to the server, alone or in combination. The image sensors 102.3 and 104.3 can be implemented using a suitably appropriate imager, such as a camera. The memories 102.4 and 104.4 can include any conventional permanent and/or temporary memory circuits or combination thereof, a non-exhaustive list of which includes Random Access Memory (RAM), Read Only Memory (ROM), Compact Disks (CD), Digital Versatile Disk (DVD), and magnetic tape.

In an example embodiment, the server 100 can store on its memory 100.2 user profiles associated with respective users. Although the user profiles are illustrated as being stored on the memory 100.2 local to the server 100, it is noted that the user profiles can alternatively be stored on a remote external memory device, for example, that is accessible by the server 100 over the network 110.

The user profiles can include, for each of a plurality of diseases a respective positivity status indicating whether the respective users with whom the respective user profiles are associated have been identified as being positive for the respective disease. The disease can be, for example, a communicable disease, for example a sexually transmittable disease. For example, a positivity status can be stored in one of the user profiles with respect to one of a plurality of predefined diseases indicating that the user is positive for the disease. For example, a positivity status can be stored in one of the user profiles with respect to one of the plurality of predefined diseases indicating that the user is negative for the disease. For example, a positivity status can be stored in one of the user profiles with respect to one of the plurality of predefined diseases indicating that no status or no current status of the user with respect to the disease has been obtained.

In an example embodiment, a value of the positivity statuses, such as positive or negative, can be obtained by the server 100 by receipt of an electronic transfer from a transmitter over the network 110. For example, FIG. 1 shows, for illustrative purposes, one such transmitter 105, but more transmitters can be communicatively coupled to the server 100 over the network 110. The electronic transfer can include or have attached thereto a data structure by which the positivity status of one or more other predefined diseases is indicated. For example, the data structure can be predefined to include a field associated with a positivity status and a disease identification according to a protocol defined for both the server 100 and the transmitter 105. Alternatively, the data structure can be in a predefined file format, e.g., a portable document format, that can be interpreted by the server 100 and upon which the processor 100.1 can perform character recognition by which the server 100 can extract from the data structure an identification of one or more of the predefined diseases and a respective positivity status associated with a respective one of the one or more of the predefined diseases. For example, processor 100.1 executes code for optical character recognition for this purpose. In an example embodiment, the data structure includes an identification of a name of the user with which the respective user profile is associated, indicating that the indicated positivity status of the disease is a positivity status of the user associated with the respective user profile.

In an example embodiment, the server 100 can verify a reliability of the transmission and its contents based on an identification of the transmitter 105. For example, in an example, the identification of the transmitter can be made based on a transmission source of the transmission. For example, in an example, the transmission can be made using an electronic mail transmission to an electronic mail recipient address of the particular user profile of the system, e.g., where each of the user profiles is assigned a respective electronic mail recipient address, so that when an electronic communication is addressed to such a respective recipient address, it is transmitted to the server 100 for processing in association with the respective user profile. Moreover, the electronic mail transmissions indicated an electronic mail sender. In an example, the server 100 may store on the memory 100.2 a directory of verified electronic mail senders, e.g., in the form of electronic mail sender addresses. These can, for example, be verified electronic sender addresses or address domains of medical entities. If the transmission is from one of these senders, the server 100 verifies the veracity of the positivity statuses indicated in the transmissions.

In an alternative example embodiment, the server 100 can verify the reliability of the transmission and its contents, for example, based on a code included in the data structure, which code is decodable to identify the transmitter 105. For example, the data structure can include therein a representation of a Quick Response (QR) code. For example, the QR code can be represented in the portable document format file.

In an example embodiment, the server 100 updates a user profile indicated by a data structure having a positivity status with respect to one of the diseases only upon satisfaction of a condition that the transmitter is verified. Alternatively, even if the transmitter is not verified, the server 100 updates the user profile with the indicated positivity status. In a particular example of this alternative embodiment, the system includes for each positivity status a flag indicating whether the respective positivity status is verified.

In an example embodiment, the server 100 can obtain the positivity statuses alternatively or additionally from transmissions from the devices, such as first device 102 and/or second device 104 on which a respective one of the user profiles is active. For example, device 102 can include an interface by which a user operating the device 102 can enter credentials associated with one of the profiles, by which an electronic session is begun in which an application running on the device 102 is logged into the respective user profile. The session can be established by communications over the network 110 between the device 102 and the server 100. While the user profile is active on the device 102, and using the application running on the device 102, the user can operate an input component of the device 102 to communicate the data structure from the device 102 to the server 100. For example, the data structure can be stored in the memory 102.4, and the data structure can be attached from the memory 102.4 to a communication made to the server 100. According to this embodiment, the positivity statuses indicated in such communications can be verified based on an included code as described above. Alternatively, the positivity statuses, when obtained from the devices 102 or 104, can be flagged as unverified.

In an example embodiment, the data structures by which the positivity statuses are provided indicate respective dates of those positivity statuses, e.g., a date of transmission from a verified transmitter or a date that a test was conducted by which the positivity status was initially determined. Accordingly, the server 100 associates the respective positivity status with the indicated date (where a date is in fact indicated). According to an example embodiment in which an unverified communication from which the positivity statuses of a user profile is populated, although a date may be indicated, the date also is considered unverified.

In an example embodiment, the server 100 maintains a clock and/or counter by which the server 100 tracks for each of the compatibility statuses for which a date has been provided a time that has lapsed since the date associated with the respective positivity status. In an example embodiment, after lapse of a predefined period of time since an indicated date of a positivity status, the server 100 clears the respective positivity status. Alternatively, after lapse of the predefined period of time, the respective positivity is not cleared but is updated to indicate that it is out of date.

In an example embodiment, the interface of the device 102 is operable for user input of a trigger to trigger the image sensor 102.3 of the device 102 to scan an image converted into the above-described data structure, e.g., into the portable document format, transmitted to the server 100, which the server 100 then processes as described above to extract the positivity statuses. For example, the server 100 can perform the optical character recognition on the data structure generated by the processor 102.1 that has processed the sensor output of the image sensor 102.3. In an alternative example embodiment, the processor 102.1 performs the optical character recognition for the application running on the device 102 to obtain the positivity statuses, with the application transmitting the described positivity status indications in association with the respective user profile active on the device 102 in a predefined data structure interpreted by the server 100 for updating the respective user profile in the memory 100.2.

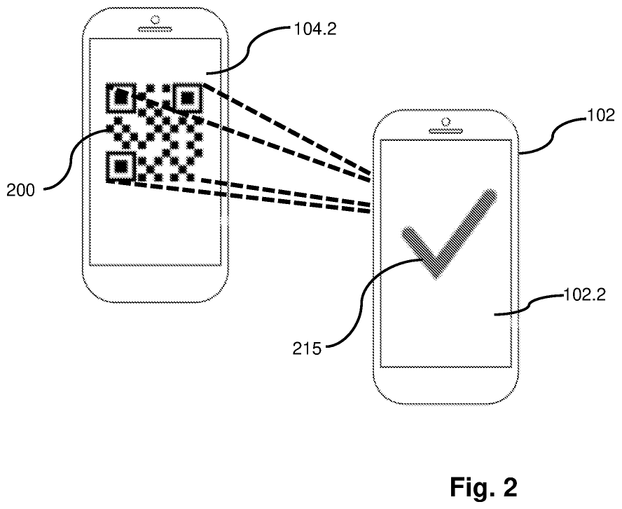

In an example embodiment, a first user of the system can operate the first device 102 running an instantiation of the application logged into a first one of the user profiles, and a second user of the system can operate the second device 104 running another instantiation of the application logged into a second one of the user profiles. The devices 102 and 104 operated by the different users are usable to provide a matching result indicating a compatibility between the users. For example, FIG. 2 shows an example of devices 102 and 104 operated in close proximity to each other. The first device 102 is operated in an active session of the first one of the profiles and second device 104 is operated in an active session of the second user profile.

In an example, the operator of the second device 104 uses an input component, e.g., a touch-sensor of the display 104.2, to provide an input that triggers the application running on the second device 104 in the active session of the second user profile to output a compatibility tag 200. In other embodiments, other input components can be used, e.g., for auditory input. The compatibility tag 200 can be, for example, a Quick Response (QR) code. In an example embodiment, the compatibility tag 200 can be generated locally by the application running on device 104. Alternatively, the application can transmit a request to the server 100 over network 110, for the processor 100.1 to generate and transmit the requested compatibility tag 200 to the device 104. In an example embodiment, the compatibility tag 200 is interpretable to extract therefrom the positivity statuses of the second user profile active on the second device 104. Alternatively, the compatibility tag 200 is interpretable to extract therefrom an identification of the second user profile, which is active on the second device 104 and for which the compatibility tag 200 is generated. It is noted that, according to the embodiment in which the compatibility tag 200 identifies the user profile without the positivity statuses being extractable therefrom, the compatibility tag 200 need not be updated over time. According to this embodiment, instead of the compatibility tag 200 being generated in response to each trigger for obtaining the compatibility tag 200, the compatibility tag 200 can be generated once and reused in response to future triggers for obtaining the compatibility tag 200. For example, the device 104 can request a compatibility tag 200 from the server 100 in response to which the server 100 generates the compatibility tag 200 from which the second user profile is identifiable, and the server 100 transmits the generated compatibility tag 200 to the device 104. The device 104 can store the compatibility tag 200 in its local memory 102.4 and can reuse the locally stored compatibility tag 200 multiple times without requiring a new updated compatibility tag 200 each time the obtaining of the compatibility tag 200 is triggered, even after passage of an extended period of time. Alternatively or additionally, the compatibility tag 200 can be stored on the memory 100.2 of the server 100 for re-sending to the device 104 in response to later requests. According to the embodiment in which the compatibility tag 200 is locally stored on the device 104, the first time the obtainment of the compatibility tag 200 is triggered, the device 104 can request the tag 200 from the server 100. Alternatively, if the compatibility tag 200 is not locally stored, in an example embodiment, the device 104 can itself generate the tag. According to an example embodiment, the compatibility tag 200 can remain in memory 104.4 as long as the session associated with the user profile is active, and can be removed from memory 104.4 in response to deactivation of the user profile on the device 104, for example, when the application running on the device 104 associated with the user profile is closed. Alternatively, the compatibility code 200 can be more permanently stored.

In response to the triggering input on device 104, device 104 outputs the compatibility code 200 that has been generated or otherwise obtained from storage. For example, FIG. 2 shows a display of compatibility code 200 on the display 104.2.

The operator of the first device 102 uses an input component, e.g., a touch-sensor of the display 102.2 or another input component, to provide an input that triggers the processor 102.1 executing the application running on the first device 102 in the active session of the first user profile to activate image sensor 102.3 to perform a scanning operation. For example, FIG. 2 shows that an image sensor 102.3 (not visible in FIG. 2) that is on a reverse side of the illustrated display 102.2 performs a scanning operation. With the devices 102 and 104 being in close proximity to each other, so that display 104.2 is in visible range of image sensor 102.3, the image sensor 102.3 performs image sensing by which device 102 obtains an image of the compatibility tag 200 displayed on display 104.2.

In an alternative example embodiment, the compatibility tag 200 is provided as a wireless data packet, e.g., using Bluetooth or Near-Field Communication (NFC). According to this alternative example embodiment, display of the compatibility tag 200 and activation of the image sensor 102.3 can be dispensed with.

In an example embodiment, the processor 102.1 of first device 102 processes the compatibility tag 200. Alternatively, the first device 102 transmits a digital representation of the compatibility tag 200 to the server 100 for processing thereof by the processor 1001.1 of server 100, the server 100 thereafter transmitting a result of the processing to the first device 102.

The processing of the compatibility tag 200 provides indications of the positivity statuses of the second profile that is active on the second device 104 which output the compatibility tag 200. For example, according to an embodiment in which the compatibility tag 200 encodes the positivity statuses, the processing of the compatibility tag 200 includes decoding tag 200, thereby directly yielding the positivity statuses. According to the embodiment in which the compatibility tag 200 identifies the second profile that is active on device 104, the processing of the compatibility tag 200 includes extracting the identification of the user profile active on the second device 104, to then obtain the positivity statuses of the decoded profile from memory 100.2 of server 100. Based on a combination of the positivity statuses obtained from the processing of the processed compatibility tag 200 and the respective positivity statuses associated with the profile associated with the first device 102, the processor 102.1 of the first device 102 or the processor 100.1 of the server 100 is configured to determine a compatibility between the two profiles.

Specifically, if the compatibility status associated with one of the two profiles concerning one of the diseases indicates being positive with respect to that disease, and the compatibility status associated with the other of the two profiles concerning that same one of the diseases indicates being negative with respect to that same disease, then the two profiles are incompatible. On the other hand if both are positive or both are negative, then the two profiles are not considered incompatible with respect to that disease because inter-person interactions between persons associated with these two profiles is not expected to cause a negative effect on either one of the two persons with respect to that disease.

In an example, the compatibility statuses are checked with respect to all of the diseases, which can be predefined at the server 100. If an incompatibility is found with respect to any one of the diseases, then the profiles are flagged as incompatible. If compatibility is found with respect to all of the diseases, then the user profiles can be considered compatible. If either of the profiles does not have a positivity status indicating positive or negative (either the positivity status is missing or the positivity field is filled with a NULL value or the like) with respect to any one of the diseases for which the other user profile has a positivity status indicating negative or for which the other user profile also does not have a positivity status indicating positive or negative (either the positivity status is missing or the positivity field is filled with a NULL value or the like), but the two user profiles are otherwise compatible, then the determined compatibility status, in an example embodiment, is set as undetermined or inconclusive.

In an example embodiment, the user profile has a setting which can be set to indicate a preference of treatment of a NULL positivity status. For example, a first user profile can be set to allow an indication of another user profile's compatibility with the first user profile as long as there is no positive positivity status, so that a NULL setting does not prevent the compatibility indication. The first user profile can also be set to instead prevent an indication of another user profile's compatibility with the first user profile if there is no negative positivity status, so that a NULL setting does prevent the compatibility indication. Similar settings can be provided with respect to whether the positivity status must be verified and/or up-to-date.

In an example embodiment, once the compatibility status is determined, the processor 102.1 of the device 102 outputs an indication of the determined compatibility of the two user profiles via an output device of the device 102. For example, the indication can be output on the display 102.2 of the device 102. For example, if the determined compatibility is that the two user profiles are compatible, the output can be a check mark 215 and/or a green mark or the like. If the determined compatibility is that the two user profiles are incompatible, the output can be an X mark, a stop sign, and/or a red mark or the like. If the determined compatibility is undetermined or inconclusive, then another respective mark can be displayed.

In an example embodiment, if a positivity status is maintained after lapse of the predefined amount of time (as discussed above) and the compatibility is determined based on a positivity status that is outdated, then the output determined compatibility can indicate this. For example, if two user profiles are compatible with respect to all diseases, except for one disease for which the determined compatibility is that there is compatibility based on an outdated negative positivity status, then the output indication of the determined compatibility can reflect that is based on an outdated status. For example, an = sign or yield or “proceed with caution” sign or the like and/or a yellow mark can be displayed.

In an example embodiment, the determined compatibility can be based entirely from the perspective of the requesting user profile. For example, in the discussed example, it is the user profile active on first device 102 that is requesting an indication of the compatibility based on a scanning of the compatibility tag 200 displayed on the display 104.2 of the second device 104. Therefore, as long as the user profile active on the second device 104 does not include a positivity status of positive for a disease for which the positivity status of the user profile that is active on the first device 102 is negative, then the output compatibility indication does not indicate a definite incompatibility. (In an example, a setting is provided by which a user profile can be set to indicate certain and in-date positivity statuses, where undetermined or outdated positivity statuses are treated as being positive. According to such an embodiment, in case of such a setting, as long as the user profile active on the second device 104 does not include a positivity status of positive or undetermined or outdated for a disease for which the positivity status of the user profile that is active on the first device 102 is negative, then the output compatibility indication does not indicate a definite incompatibility.)

According to this embodiment, for two-way compatibility to be provided, the described process would be performed twice. First one of the devices 102/104 would be controlled to display the compatibility tag 200 and the other of the devices 102/104 would be controlled to scan the compatibility tag 200 and display the compatibility indication, and then the other of the devices 102/104 would be controlled to display the compatibility tag 200 and the one of the devices 102/104 would be controlled to scan the compatibility tag 200 and display the compatibility indication. In this way, each device 102/104 displays a respective compatibility indication from its respective perspective.

If a first user profile has a positivity status that is positive for a disease for which the positivity status of a second user profile, against which the first profile is being matched, is negative and this results in a compatibility indication indicating that the two user profiles are not a match, this can indicate to the user associated with the second profile that the user associated with the first user profile is positive for a communicable disease. The user associated with the first user profile of course need not share this by simply not providing display of a compatibility tag 200 for scanning by the device of the other user. However, the user might want to provide the compatibility tag 200 on the chance that a match will be found between the two user profiles. Subsequently, however, when the output indication is that there is no match and the two user profiles are incompatible, this can cause embarrassment to the user whose user profile has the positivity status indicated the user is positive with respect to the respective disease, although the user associated with the other user profile might not be able to determine which one or more diseases is the one whose positivity status is positive.

In an example embodiment, the system is configured to provide a setting that can mitigate against such potential embarrassment. For example, according to one setting, how the positivity statuses of the two profiles match up can be the entire basis for the determination of compatibility of the two user profiles, the indications for which are ultimately output. According to another setting, compatibility is determined based on the positivity statuses with respect to the diseases and also based on other compatibility factors. For example, compatibility can be further based on user-indicated likes, ages, preferences, professions, residence locations, desired residence locations, behavioral characteristics, etc. for which compatibility can be algorithmically determined. In an example in which such a setting is provided, if an output indication is that two user profiles are not a match, it would not be known if the reason for the indicated incompatibility is due to a positive status with respect to a disease or because of another factor.

In an example embodiment of the present invention, the system executes an algorithm for continuous unprompted searches for pairwise matches between any two of a plurality of user profiles stored in the memory 100.2 of the server 100. The algorithmic matching is performed by the processor 100.1 of the server 100 based on respective sets of characteristics identified in respective ones of the user profiles. For example, such characteristics can include likes, ages, goals, preferences, professions, residence locations, desired residence locations, desired vacation destinations, behavioral characteristics, etc. for which compatibility between different pairwise combinations of the stored user profiles can be algorithmically determined.

If the server 100 finds a pairwise match of two of the user profiles, the server 100 pushes notifications of the match to each of the user profiles. When a user has an instantiation of the application running on one of the devices, e.g., devices 102 and 104, which can be in the form of a smart phone, tablet, laptop, personal computer, or the like, with one of the matched user profiles being active in the instantiation, the notification is output via an output component of the device. For example, notification can be via a speaker with a particular ring tone or can take the form of a pop-up on a display screen or can be an indicator of presence of a new message in an in-box that can be accessed using the device 102/104 via the application or alternatively even in a message program that operates independently of the application, such as an e-mail inbox. The notification to one of the matched user profiles provides information regarding the other user profiles of the matched pair, and vice versa. In an example, the notification is accompanied with a widget by which input can be provided for indicating whether a connection is sought by the notified user profile with the other user profile of the matched pair. If indications for a sought connection are input using each of the profiles, then the system provides access for each user profile to message the other user profile.

However, the algorithm further conditions the pushing of the notifications of the match based on the positivity statuses of the user profiles as discussed with respect to the previously described embodiments. For example, if two of the user profiles are otherwise algorithmically matched, but are determined to be incompatible with respect to the positivity statuses, such as where one of the user profiles includes a positivity status for one of the diseases that is negative for that disease and another of the user profiles includes a positivity status for that same one of the diseases that is positive for that disease, then the system ignores the match and does not push a notification of the match to the user profiles. According to an example embodiment, the system only pushes notifications where all positivity statues are confirmed updated statuses. According to an alternative example embodiment, the system pushes notifications as long as there are no confirmed incompatibilities, so that, for example, an outdated negative positivity status does not prevent the pushing of the match notification because of the outdatedness of the positivity status and/or an unprovided positivity status for a disease does not prevent the pushing of the match notification because of the lack of the positivity status. According to a variant of this example embodiment, notification is provided with a further indicator that further information may be advised. According to yet another example embodiment, a user operating an active user profile can set a setting of the user profile indicating whether unverified and/or outdated positivity statuses can be relied upon. For example, the setting can be set so that only verified and up-to-date statuses can be used for matching, in which case, if the user profile for which the setting has been set includes a negative positivity status for a particular disease, then, for another user profile to be matched to this set user profile, the other user profile must include for that disease a verified and up-to-date negative positivity status.

In an example embodiment, the user profile can also be set in the reverse manner, in which only if another user profile's positivity status for a disease is positive, will it prevent the match to the first user profile for which the setting has been set (and for which disease the positivity status of the first user profile is negative). For example, a NULL positivity status or an unverified negative status would not prevent the match.

It is noted that although, according to the embodiments in which notifications are pushed regarding the unprompted match searching and compatibility determination are described as occurring in two steps, in which first a match is found and then an incompatibility determination is performed for conditioning the pushing of the notification of the found match, in an alternative embodiment the compatibility determination can be performed as part of the algorithmic matching.

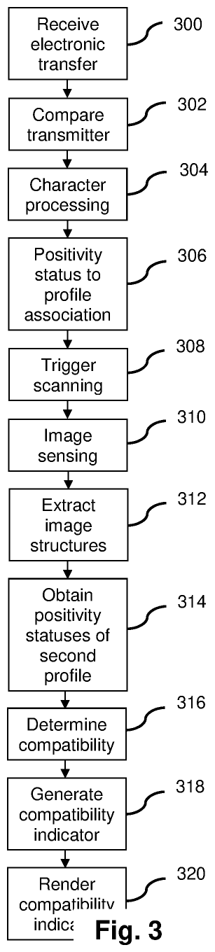

FIG. 3 illustrates a compatibility determination method according to an example embodiment of the present invention. At step 300, the processor 100.1 of the server 100 receives from a transmitter, e.g., the transmitter 105, and via the network 110 at least one electronic transfer having attached thereto a data structure. The electronic transfer includes an identification of the transmitter 105. The receipt of the electronic transfer is made in association with a first user profile.

At step 302, the processor 100.1 compares the identified transmitter 105 to a predetermined set of transmitters, for example as stored in memory 100.2, thereby determining that the transmitter 105 is included in the set of transmitters.

At step 304, the processor 100.1 performs a character processing of the attached data structure, thereby identifying in the data structure one or more of the plurality of predefined diseases and, for each of the identified one or more of the plurality of predefined diseases, a respective positivity status.

At step 306, based on the determination of the inclusion of the identified transmitter 105 in the set of transmitters, for each of the one or more of the plurality of diseases, the processor 100.1 associates the respective identified positivity status for the respective one of the diseases with the first user profile.

Subsequent to the association of the positivity statuses for respective diseases with the first user profile, and responsive to a scan trigger on a device, e.g., the first device 102, on which the first user profile is active, at step 308, the first device 102 activates the image sensor 102.3 of the device 102 to scan a first compatibility tag. The first compatibility tag can be displayed, for example, on the display 104.2 of the second device 104.

At step 310, the image sensor 102.3 performed image sensing to obtain an image. At step 312, the processor 102.1 of the first device 102 or the processor 100.1 of the server 100 processes the image to extract structures from the image. For example, the system processes the image to thereby recognize the first compatibility tag.

At step 314, the processor 102.1 of the first device 102 or the processor 100.1 of the server 100 processes the scanned first compatibility tag thereby obtaining a respective positivity status for each of the plurality of diseases, which are associated with the second user profile active on the second device 104. The obtained positivity status can be a negative status, a positive status, or an unavailable or NULL status. The obtained positivity statuses can be further specified as being up-to-date or outdated.

At step 316, the processor 102.1 of the first device 102 or the processor 100.1 of the server 100 determines a compatibility between the first and second user profiles based on a combination of the positivity statuses obtained from the processing of the scanned first compatibility tag and the respective identified positivity status for the one or more of the plurality of diseases associated with the first user profile. At step 318, the first device 102 or the server 100 generates a compatibility indicator indicating the determined compatibility. At step 320, the first device 102 renders the compatibility indicator on the display 102.2 of the first device 102.

FIG. 4 illustrates a compatibility determination method triggered by a user of, for example, the first device 102 providing input that activates the image sensor 102.3 of the first device 102 to perform scanning by taking an image, according to an example embodiment of the present invention. For example, the first user profile is active on the first device 102 running an instantiation of a compatibility determination application.

At step 400, a user uses an input component of the first device 102 for generating a first scan trigger. In response to the first scan trigger on the first device 102, on which a first user profile is active, the processor 102.1 of the device 102 activates the image sensor 102.3 to record an image. At step 402, the processor 102.1 of the device 102 or the processor 100.1 of the server 100 (after transmission of the image sensed by the image sensor 102.3) processes the image, thereby extracting one or more of the plurality of predefined diseases and, for each of the one or more of the plurality of predefined diseases, a respective positivity status. At step 404, in response to the extraction, for each of the one or more of the plurality of diseases, the respective identified positivity status for the respective one of the diseases is associated with the first user profile on the central server 100.

Subsequent to population of the positivity statuses of the first user profile, a user uses the input component of the first device 102 (or of another device on which the first user profile is active) for generating a second scan trigger. In response to the second scan trigger, at step 406, the processor 102.1 of the first device 102 (or other device) activates the image sensor 102.3 of the first device 102 (or other device) to scan a compatibility tag, e.g., displayed on display 104.2 of the second device 104.

At step 408, the processor 102.1 of the first device 102 or the processor 100.1 of the server 100 processes the scanned compatibility tag, thereby obtaining a respective positivity status for each of the plurality of diseases of the second user profile for which the compatibility tag has been generated, e.g., by an instantiation of the application running on the second device 104 with the second user profile being active thereon.

At step 410, based on a combination of the positivity statuses obtained from the processing of the scanned compatibility tag and the respective identified positivity statuses for the one or more of the plurality of diseases associated with the first user profile, the processor 102.1 of the first device 102 (or other device that had scanned the compatibility tag) or the processor 100.1 of the server 100 generates a compatibility indicator. At step 412, the processor 102.1 renders the compatibility indicator on the display 102.2 of the first device 102 or other device.

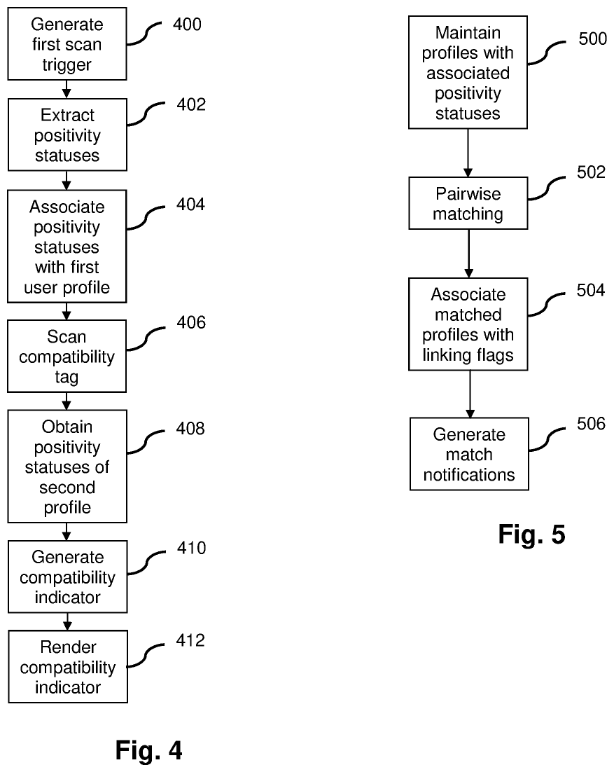

FIG. 5 illustrates a method for proactive and continuous pairwise match searching according to an example embodiment of the present invention. At step 500, the server 100 maintains in its memory device 100.2 a plurality of user profiles and, for each of the user profiles, associates in the memory device 100.2 positivity statuses for respective ones of the plurality of predefined diseases.

At step 502, subsequent to the associations, the processor 100.1 of the central server 100, performs pairwise matching of respective pairs of the plurality of user profiles at least partially based on the positivity statuses associated with the plurality of user profiles. For each of the pairs that are matched, the pairing is made conditional upon that the positivity statuses associated with either one of the user profiles of the pair do not include a positive status for any disease for which a negative status is associated with the other user profile of the pair. If the positivity statuses of both user profiles for a particular disease indicate that both are positive or that both are negative, those positivity statuses do not prevent the match.

If a first one of the user profiles has a setting that allows for NULL statuses to be treated as a negative, then another user profile can be matched to that first user profile even if no positivity status has been provided in the other user profile for a disease for which the positivity status of the first user profile is negative. In an example, a first user profile can be set to treat its own NULL status as negative and a second user profile's NULL status as positive, so that if both user profiles have a NULL status for a particular disease, the second user profile will not be indicated as a match to the first user profile. In example embodiments in which the system performs proactive continuous matching, if a first user profile cannot be treated as a match to a second user profile, then the second user profile will also not be indicated to the first user profile as being a match even if settings of the first user profile allow the second user profile to be considered a match.

After a match is found between two user profiles, at step 504, the processor 100.1 of the server 100, for each of the found pairs, associates each one of the user profiles of the pair with a respective electronic flag linking the other user profile of the pair as a match. The electronic flag is an electronic reference to the other user profile of the pair. At step 506, the processor 100.1 of the server 100 generates respective notifications that it provides to the respective user profiles of the respective pair notifying of the match to the other user profile of the pair. Such a notification can be sent to an inbox associated with the respective user profiles, such as an electronic mail inbox. Alternatively or additionally, the notifications can be output on an output component of a respective device on which a respective one of the user profiles is active, e.g., immediately or, if no application is currently running with the respective user profile being active, then at a such later time when the application is running with the user profile being active. For example, the notification can be output as an of the other user profile as being matched on the display 102.2 of the first device 102.

The example embodiments described and shown in the figures have been chosen only as examples. Different example embodiments can be combined with each other in their entirety or with regard to individual features. One example embodiment can also be supplemented with features of a further example embodiment.

In addition, method steps according to the present invention can be repeated, and can be carried 10 out in a sequence differing from that described.

If an example embodiment contains an “and/or” linkage between a first feature and a second feature, this is to be read as meaning that according to one example embodiment, both of the first and second features are included, and according to another example embodiment, only one of the first and second features are included.

What is claimed is:

1. A method comprising:
receiving, by a processor and from a transmitter via a network, at least one electronic transfer having attached thereto a data structure, the electronic transfer including an identification of the transmitter, the receipt being made in association with a first user profile;
comparing, by the processor, the identified transmitter to a predetermined set of transmitters, thereby determining that the transmitter is included in the set of transmitters;
performing a character processing of the attached data structure, thereby identifying in the data structure one or more of a plurality of predefined diseases and, for each of the one or more of the plurality of predefined diseases, a respective positivity status;
conditional upon the determination of the inclusion of the identified transmitter in the set of transmitters, for each of the one or more of the plurality of diseases, associating the respective identified positivity status for the respective one of the diseases with the first user profile on a central server; and
subsequent to the association and responsive to a scan trigger on a device on which the first user profile is active:
the device activating an image sensor of the device to scan a first compatibility tag;
processing the scanned first compatibility tag, by the device or by the central server, thereby obtaining a respective positivity status for each of the plurality of diseases; and
based on a combination of the positivity statuses obtained from the processing of the scanned first compatibility tag and the respective identified positivity status for the one or more of the plurality of diseases associated with the first user profile, generating, by the device or the server, a compatibility indicator and rendering the compatibility indicator on a display of the device.

2. The method of claim 1, further comprising the server associating the receipt with the first user profile based on the transmission being addressed with an electronic recipient address that is associated with the first user profile.

3. The method of claim 1, wherein the first compatibility tag is a Quick Response (QR) code.

4. The method of claim 1, further comprising, subsequent to the association:
generating, by the device on which the user profile is active or by the server, a second compatibility tag, wherein the second compatibility tag is processable by a second device to obtain, by the second device, the respective positivity status for the one or more diseases associated with the first user profile; and
rendering the second compatibility tag on the display of the device.

5. The method of claim 4, wherein the generating and rendering of the second compatibility tag occurs responsive to the scan trigger.

6. The method of claim 4, wherein the generating and rendering of the second compatibility tag occurs responsive to a tag generating trigger separate from the scan trigger.

7. The method of claim 1, wherein the compatibility indicator is set to positive, the setting being made conditional upon that the positivity statuses obtained by the processing of the scanned first compatibility tag do not include a positive status for any disease for which a negative status is associated with the first user profile.

8. The method of claim 1, wherein the compatibility indicator is set to positive, the setting being made conditional upon that the positivity statuses obtained by the processing of the scanned first compatibility tag do not include a positive status for any disease for which a positive status is not associated with the first user profile.

9. The method of claim 1, wherein the compatibility indicator is set to negative, the setting being made conditional upon that the positivity statuses obtained by the processing of the scanned first compatibility tag include a positive status for at least one disease for which a negative status is associated with the first user profile.

10. The method of claim 1, wherein the compatibility indicator is set to negative, the setting being made conditional upon that, for each of at least one disease for which a negative status is associated with the first user profile, the positivity statuses obtained by the processing of the scanned first compatibility tag either include a positive status or do not include a negative status.

11. The method of claim 1, wherein the compatibility indicator is set to neutral, the setting being made in response to satisfaction of a condition that, for each of at least one disease for which a negative status is associated with the first user profile, the positivity statuses obtained by the processing of the scanned first compatibility tag is that a status has not been provided.

12. The method of claim 1, wherein the compatibility indicator is set to neutral, the setting being made in response to satisfaction of a condition that, for each of at least one disease for which a negative status is associated with the first user profile, the positivity statuses obtained by the processing of the scanned first compatibility tag include a negative status associated with a date that is prior to a current date by a time period that is greater than a predefined time period to which the device compares the time period of the associated date.

13. The method of claim 1, wherein, based on the processing of the scanned first compatibility tag, a respective date is obtained by the device of the central server in association with each of the respective positivity statuses obtained from the processing of the scanned first compatibility tag, wherein the generation of the compatibility indicator is based on, for each of at least one of the respective dates, a comparison of a time period from the respective date until a current date to a predefined time period.

14. A method comprising:
in response to a first scan trigger on a first device on which a first user profile is active:
activating, by the device, an image sensor to record an image;
processing, by the device or by a central server to which the device is communicatively coupled, the image, thereby extracting one or more of a plurality of predefined diseases and, for each of the one or more of the plurality of predefined diseases, a respective positivity status; and
in response to the extraction, associating for each of the one or more of the plurality of diseases, the respective identified positivity status for the respective one of the diseases with the first user profile on the central server; and
subsequent to the association and responsive to a second scan trigger on the first device on which the first user profile is active or on a second device on which the first user profile is active:
the first or second device activating an image sensor of the respective first or second device to scan a first compatibility tag;
processing the scanned first compatibility tag, by the respective first or second device or by the central server, thereby obtaining a respective positivity status for each of the plurality of diseases; and
based on a combination of the positivity statuses obtained from the processing of the scanned first compatibility tag and the respective identified positivity status for the one or more of the plurality of diseases associated with the first user profile, generating, by the respective first or second device or by the server, a compatibility indicator and rendering the compatibility indicator on a display of the respective first or second device.

15. The method of claim 14, wherein:
the processing of the image includes extracting an identification of an entity;
the method further comprises comparing, by the device or the server, the identified entity to a predetermined set of entities, thereby determining that the identified entity is included in the set of entities; and
the associating of the respective identified positivity status for the each respective one of the diseases with the first user profile on the server is performed conditional upon the determination of the inclusion of the identified entity in the set of entities.

16. A system comprising:
a central server; and
a first device that is communicatively coupled to the central server and that includes an image sensor, a display, and a processor;
wherein:
the central server is configured to:
receive, from a transmitter and via a network, at least one electronic transfer having attached thereto a data structure, the electronic transfer including an identification of the transmitter, the receipt being made in association with a first user profile;
compare the identified transmitter to a predetermined set of transmitters, thereby determining that the transmitter is included in the set of transmitters;
perform a character processing of the attached data structure, thereby identifying in the data structure one or more of a plurality of predefined diseases and, for each of the one or more of the plurality of predefined diseases, a respective positivity status; and
conditional upon the determination of the inclusion of the identified transmitter in the set of transmitters, for each of the one or more of the plurality of diseases, associate the respective identified positivity status for the respective one of the diseases with the first user profile;
subsequent to the association and responsive to a scan trigger on the first device on which the first user profile is active by a communication of the device with the server, the processor of the first device is configured to activate the image sensor of the first device to scan a first compatibility tag;
the central server or the processor of the first device is configured to process the scanned first compatibility tag, thereby obtaining a respective positivity status for each of the plurality of diseases;
based on a combination of the positivity statuses obtained from the processing of the scanned first compatibility tag and the respective identified positivity status for the one or more of the plurality of diseases associated with the first user profile, the processor of the first device or the server is configured to generate a compatibility indicator; and
while the first user profile is active on the first device, the processor is configured to render the compatibility indicator on the display of the first device.

17. A method comprising:
in response to an obtainment trigger on a first device on which a first user profile is active:
processing, by the device or by a central server to which the device is communicatively coupled, a data structure, thereby extracting one or more of a plurality of predefined diseases and, for each of the one or more of the plurality of predefined diseases, a respective positivity status; and
in response to the extraction, associating, for each of the one or more of the plurality of diseases, the respective identified positivity status for the respective one of the diseases with the first user profile on the central server; and
subsequent to the association and responsive to a scan trigger on the first device on which the first user profile is active or on a second device on which the first user profile is active:
the first or second device activating an image sensor of the respective first or second device to scan a first compatibility tag;
processing the scanned first compatibility tag, by the respective first or second device or by the central server, thereby obtaining a respective positivity status for each of the plurality of diseases; and
based on a combination of the positivity statuses obtained from the processing of the scanned first compatibility tag and the respective identified positivity status for the one or more of the plurality of diseases associated with the first user profile, generating, by the respective first or second device or by the server, a compatibility indicator and rendering the compatibility indicator on a display of the respective first or second device.

18. A method comprising:
for each of one or more of a plurality of diseases, associating, by a central server, a respective positivity status for the respective disease with a first user profile; and
subsequent to the association and responsive to a scan trigger on a device on which the first user profile is active and that is communicatively coupled to the central server:
the device activating an image sensor of the device to scan a first compatibility tag;
processing the scanned first compatibility tag, by the device or by the central server, thereby obtaining a respective scanned positivity status for each of the plurality of diseases; and
based on a combination of the positivity statuses obtained from the processing of the scanned first compatibility tag and the respective positivity status for the one or more of the plurality of diseases associated with the first user profile, generating, by the device or the central server, a compatibility indicator and rendering the compatibility indicator on a display of the device.

19. A method comprising:
for each of a plurality of user profiles, associating, by a central server and with the respective user profile, a positivity status for each disease of a respective subset of a plurality of diseases;
subsequent to the associations, performing, by the central processor, pairwise matching of respective pairs of the plurality of user profiles at least partially based on the positivity statuses associated with the plurality of user profiles, wherein, for each of the pairs that are matched, the pairing is made conditional upon that the positivity statuses associated with either one of the user profiles of the pair do not include a positive status for any disease for which a negative status is associated with the other user profile of the pair;
for each of the respective pairs:
associating each one of the user profiles of the pair with a respective electronic flag linking the other user profile of the pair as a match; and
responsive to the associations with the respective electronic flags, generating on an output component of a respective device on which a respective one of the user profiles is active, an indicator of the other user profile or the pair as a match.
